(19) United States
(12) Patent Application Publication
(10) Pub. No.: US 2008/0299638 A1
(43) Pub. Date: Dec. 4, 2008

(54) Pharmaceutical Composition and Method of Treating Hepatitis with Arginases

(76) Inventor: Ning Man CHENG, Hong Kong (CN)

Correspondence Address: EAGLE IP LIMITED, 22/F., KWAI HUNG HOLDINGS CENTRE, 89 KING'S ROAD, NORTH POINT, HK

(21) Appl. No.: 11/680,631

(22) Filed: Mar. 1, 2007

Related U.S. Application Data
(63) Continuation of application No. PCT/CN05/01411, filed on Sep. 6, 2005.

(30) Foreign Application Priority Data
Sep. 8, 2004 (CN) 200410076854.4

(51) Int. Cl. C12N 9/96; C12N 9/78

(52) U.S. Cl. 435/188; 435/227

(57) ABSTRACT

The invention discloses methods for treating hepatitis with human arginase I modified by polyethylene glycol and uses of it in manufacturing of a medicament.

### FIELD OF INVENTION

The present invention is related to pharmaceutical composition and use therefor. In a preferred embodiment, the present invention is related to pharmaceutical composition that is capable to treat hepatitis.

### BACKGROUND OF INVENTION

There are many antiviral drugs for the treatment of hepatitis, the following are the most frequently used: (1) Interferon: a broad-spectrum antiviral agent which induces cells to produce their own antiviral protein through the reaction to the cell surface receptors rather than directly killing or suppressing virus and therefore lead to the suppression of hepatitis B and C virus replication. At the same time it boosts the activity of NK cells, macrophages and T-lymphocytes, modulates immune system and enhances antiviral ability. (2) Interleukin-2: a T-cell growth factor, which modulates immune system and possesses antivirus and anti-tumor ability. (3) Nucleosides: Acyclovir, for example, is an acyclic purine nucleoside which suppresses the replication of various DNA virus. (4) Arabinoside: proved to be potentially effective against hepatitis B both in vivo and in vitro. Some patients show HBV DNA polymerase latency with improved abnormal biochemistry and liver biopsy during treatment. (5) Others: Hepatocyte growth-promoting factor (pHGF), thymosin, anti-hepatitis B ribonucleic acid, ribavirin, levamisole, lentinan, potenline, phytohemagglutinin and etc. However, the effectiveness of the aforesaid drugs is unsatisfying and they are easy to induce adverse side-effect.

### SUMMARY OF INVENTION

In the light of the foregoing background, it is an object of the present invention to provide a more effective pharmaceutical composition for treating hepatitis. In a preferred embodiment, pharmaceutical compositions are provided for selectively reducing arginine level of a patient in the treatment of hepatitis.

Accordingly, in one aspect, an enzyme which degrades arginine (arginine degrading enzyme) is provided for the preparation of medicament. In a preferred embodiment, the arginine degrading enzyme is arginase or arginine deiminase. Yet another embodiment, the arginine degrading enzyme is an isolated and substantially purified recombinant arginase. In a more preferred embodiment, the arginase of the present invention is human arginase I. Yet another more preferred embodiment, the human arginase I of the present invention substantially comprises the same nucleic acid sequence as set forth in SEQ ID NO: 1 or SEQ ID NO: 2. and said nucleic acid sequences comprise the same amino acid sequence as set forth in SEQ ID NO: 3. Yet another embodiment, the recombinant human arginase I of the present invention is of 80-100% purity. In a more preferred embodiment, recombinant human arginase I of the present invention is of 90-100% purity.

Yet another preferred embodiment, the arginase of the present invention is modified to have sufficiently high enzymatic activity and stability to maintain “adequate arginine deprivation” (hereinafter referred to as “AAD”) in a patient for at least 3 days. One preferred method of modification is an amino-terminal tag of six-histidine. Yet another preferred modification is pegylation to increase the stability of the enzyme and minimize immunoreactivity elicited by the patient thereto. In another more preferred embodiment, the pegylation comprises a coupling agent covalently bond to at least one polyethylene glycol. In a most preferred embodiment, the coupling agent is 2,4,6-trichloro-s-triazine (cyanuric chloride, CC) or succinimide propionic acid (SPA). The modified arginase has specific activity of at least 250 I.U./mg. In one preferred embodiment the specific activity is of at least 300-350 I.U./mg. In a most preferred embodiment, the specific activity is of at least 500 I.U./mg. In another preferred embodiment, said arginase is modified to have sufficient stability and to have a plasma or serum half-life of at least approximately 3 days.

Yet another preferred embodiment, the medicament prepared by the present invention is provided to treat hepatitis. In a more preferred embodiment, the medicament prepared by the present invention is provided to treat hepatitis B.

In another implementation, there are further provided pharmaceutical composition comprising isolated and substantially purified recombinant arginase. In a preferred embodiment, the pharmaceutical composition provided therein comprising recombinant arginase with 80-100% purity. Yet another preferred embodiment, recombinant human arginase is any arginine degrading enzyme, for example arginine deiminase or human arginase I. In the most preferred embodiment, said enzyme comprises essentially of the same amino acid sequence as set forth in SEQ ID NO: 1 or SEQ ID NO: 2 and said amino acid coding sequences comprise the same amino acid sequence as set forth in SEQ ID NO: 3. In a preferred embodiment, said arginase is modified to have high specific activity and sufficient stability in patient's plasma or serum half-life for approximately 3 days. Another preferred modification is pegylation to increase the stability of the enzyme and minimize immunoreactivity

Yet another aspect of the present invention, a pharmaceutical composition is provided to lower arginine level of a patient. In one preferred embodiment, the present invention is to modulate hepatitis. In a more preferred embodiment, the present invention is capable to treat hepatitis B. In another embodiment, pharmaceutical composition of the present invention is prepared in the form of solid, liquid, emulsion, suspension, small albumin aggregate (SAA) or liposome. Yet another preferred embodiment, the pharmaceutical composition of the present invention is suitable to administrate orally or intravenously.

### BRIEF DESCRIPTION OF FIGURES

FIGS. 1A, 1B and 1C are the nucleic acid sequence of human arginase I and the corresponding amino acid sequence.

FIG. 1A is the nucleic acid sequence (SEQ ID NO: 1) from EcoRI/MunI to XbaI sites of plasmid pAB101. Nucleic acid (nt)1-6, EcoRI/MunI site; nt 481-486, region −35 of promoter 1; nt 504-509, region −10 of promoter 1; nt 544-549, region −35 of promoter 2; nt 566-571, region −10 of promoter 2; nt 600-605, ribosome binding site; nt 614-616, start codon; nt 632-637, NdeI site; nt 1601-1603, stop codon; nt 1997-2002, XbaI site.

FIG. 1B is the nucleic acid sequence (SEQ ID NO: 2) of the modified human arginase and its corresponding amino acid sequence (SEQ ID NO: 3). Nucleic acids 614-1603 in FIG. 1A are the coding region of the modified arginase amino acid sequence. The six histidine (SEQ ID NO: 4) on the N-terminal are shown underlined. Translational stop codons are marked with *.

FIG. 1C is the nucleic acid sequence (SEQ ID NO: 8) of normal human arginase I and its corresponding amino acid sequence (SEQ ID NO: 9).

### DETAILED DESCRIPTION OF THE PREFERRED EMBODIMENTS

As used herein, the term “pegylated Arginase” refers to Arginase I of present invention modified by pegylation (see WO2004/001048) to increase the stability of the enzyme and minimize immunoreactivity.

As used herein, the phrase “substantially the same”, whether used in reference to the nucleotide sequence of DNA, the ribonucleotide sequence of RNA, or the amino acid sequence of protein, refers to sequences that have slight and non-consequential sequence variations from the actual sequences disclosed herein. Species with sequences that are substantially the same are considered to be equivalent to the disclosed sequences and as such are within the scope of the appended claims. In this regard, “slight and non-consequential sequence variations” means that sequences that are substantially the same as the DNA, RNA, or proteins disclosed and/or claimed herein are functionally equivalent to the sequences disclosed and/or claimed herein. Functionally equivalent sequences will function in substantially the same manner to produce substantially the same compositions as the nucleic acid and amino acid compositions disclosed and claimed herein. In particular, functionally equivalent DNAs encode proteins that are the same as those disclosed herein or proteins that have conservative amino acid variations, such as substitution of a non-polar residue for another non-polar residue or a charged residue for a similarly charged residue. These changes include those recognized by those of skill in the art not to substantially alter the tertiary structure of the protein. The term “sufficiently high enzymatic activity” refers to the enzyme specific activity of the recombinant human arginase for at least 250 I.U./mg, preferably at least 300-350 I.U./mg, more preferably at least 500 I.U./mg. In the preferred embodiment, the arginase has a specific activity of 500-600 I.U./mg. The term “stability” refers to in vitro stability of the arginase. More preferably, the stability refers to in vivo stability. The rate of decrease of enzyme activity is inversely proportional to the plasma stability of the isolated, purified recombinant human arginase. This relationship is reflected in the half-life of human arginase in plasma.

As used herein, the term “adequate arginine deprivation” (AAD) refers to in vivo arginine level at or below 10 μM. The term “half-life” (½-life) refers to the time that would be required for the concentration of the arginase in human plasma in vitro, to fall by half.

All other information about the technical know-how and terms as used herein can be found in WO2004/001048 and WO2004/000349.

In order to investigate the anti-hepatitis B virus effect of arginase, the present invention uses hepatitis B viral gene transfected human liver cancer cell line 2.2.15 to test the cellular toxicity of arginase, suppression of HBsAg and HBeAg secretion by arginase and the suppression of HBV-DNA by arginase. Comparison is done using lamivudine by GlaxoWellcome, UK as a positive control. The result shows that: TC50 of pegylated recombinant arginase after 8 days of CPE method drug addition is 40 IU/ml, TC0 is 20±0 IU/ml. The percent suppression of HBeAg secretion, IC50 and SI from two batches of experiments using TC0=20 IU/ml is 68.69±8.89, 6.37±0.45 IU/ml, 6.30±0.45 respectively. The percent suppression of HBsAg secretion, IC50 and SI are 29.81±27.35, 10.72 IU/ml (from one batch of experiment) and 3.73 (from one batch of experiment) respectively. The IC50 of HBV-DNA dot blotting in the supernatant of the culture medium is 13.18±0.45 IU/mL, selective index (SI) is 3.19±0.98. The IC50 of HBV-DNA Southern Blot Sum in cell is 19.79±7.95 IU/ml, selective index is 2.91±0.88. The IC50 of HBV-DNA Southern Blot In Lane in cell is 20.06±1.96 IU/ml, selective index is 2.00±0.20. The TC50 and TC0 of positive control lamivudine are 1198.97±97.50 and 800±0 μg/ml respectively. The HBeAg and HBsAg secretion of 2.2.15 cells are not significantly suppressed after incubating with TC0 800 μg/ml lamivudine for 8 days. The IC50 of HBV-DNA dot blotting in the supernatant of the culture medium is 113.76 μg/mL, selective index is 10.54. The IC50 of HBV-DNA Southern Blot Sum in cell is 88.78±6.37 μg/mL, selective index is 13.54±0.97. The multiple experimental results are consistent with published literature, indicating that the experiments are reliable. The result shows that: Arginase significantly inhibits the secretion of HBsAg and HBeAg and lowers HBV-DNA in cells.

### Example 1

### Preparation of Materials

1.1 Drug to be Tested

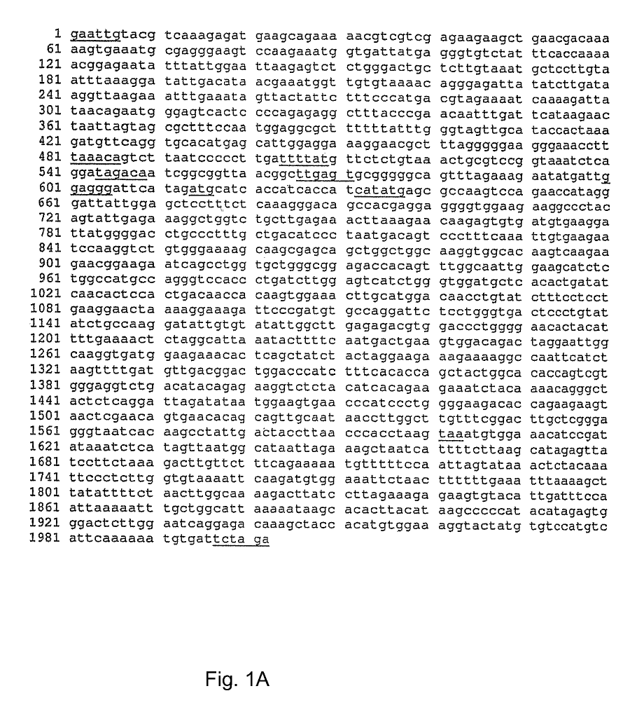

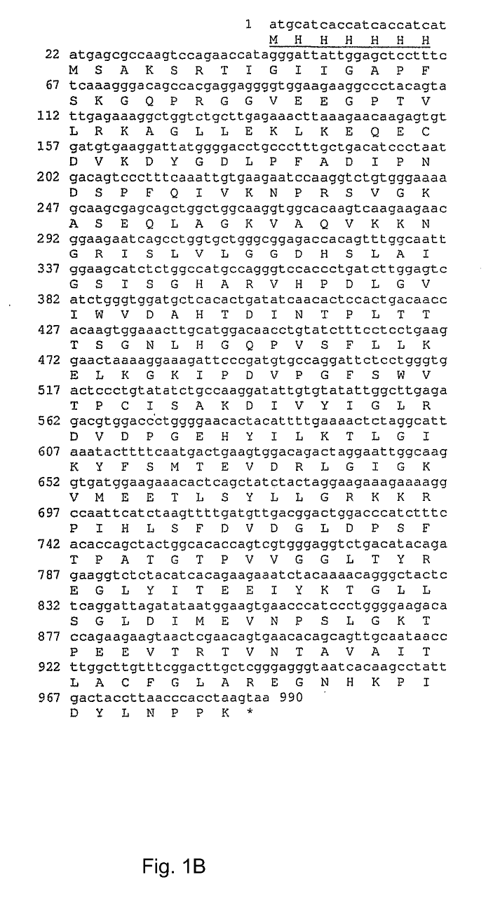

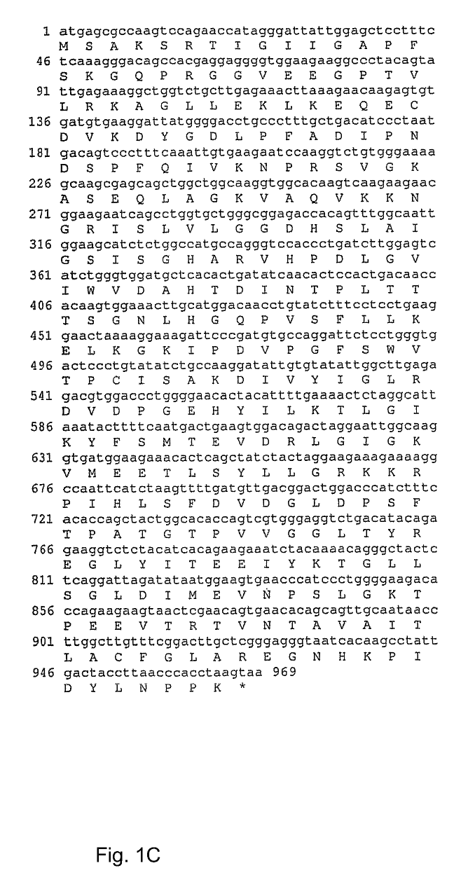

Name: Pegylated recombinant human arginase (BCT-100), hereinafter “arginase”. Said arginase comprises nucleic acid sequence as shown in FIGS. 1A, 1B and 1C and its corresponding amino acid sequence.

Preparation: Please refer to example 1-8 in specification of WO2004/001048. Recombinant human arginase can be obtained from Professor Ikemoto Masaki's laboratory prior to the earliest application date of WO2004/001048 (University of Kyoto; Address: 53 Kawahara-cho, Shogoin, Sakyo-ku, Kyoto-shi, Kyoto 606-8507 Japan). Arginases are prepared by MEM medium according to the designated dosage groups.

Preservation: store in 4° C. refrigerator.

1.2 Positive control: lamivudine, produced by GlaxoWellcome, UK. Batch No.: B008923, 100 mg per tablet, drug is soaked and dissolved in medium, centrifuge to remove sediments, prepared by MEM medium according to the designated dosage groups during experiment. Preservation: store in 4° C. refrigerator.

1.3 2.2.15 cell: 2.2.15 cell line of human liver cancer cell (Hep G2) transfected with Hepatitis B virus, constructed by Mount Sinai Medical Center. Imported and cultivated by our laboratory.

1.4 Reagents: Eagles MEM powder, G-418 (Geneticin), yeast t-RNA, proteinase-K, by Gibco, U.S.A.; fetal bovine serum, by Hyclone Lab, U.S.A.; L-glutamine, Jingke Chemical Reagent Company; HBsAg, HBeAg radioimmunoassay, China Isotope Corporation Beifang Immunoreagent Research Center; kanamycin, North China Pharmaceutical Group Corporation; polyethylene glycol, Fluka, Sweden; DMSO, Sigma; d-³²p-dCTP, Beijing Yahui Bio Medical Engineering Company;

1.5 Instruments: culture bottle, Tunclon™, Denmark; 96-well, 24-well and 6-well plates, Corning, U.S.A.; Carbon Dioxide incubator, Shel-Lab, U.S.A.; γ-counter, Beckman, Germany; Scanner, Microtek; gel-pro analyzer software, MEDIA Cybemetice®;

1.6 Cell Culture Medium and Reagent

MEM medium 100 ml: containing fetal bovine serum 10%, glutamine 0.03%, G418 380 μg/ml, kanamycin 50 μg/ml.

1.7 2.2.15 cell culture: add 0.25% Trypsin into culture bottle with fully grown 2.2.15 cells, digest 10 minutes at 37° C., add medium to disperse, 1:3 subculture, full grown after 10 days.

### Example 2

### Test for Arginase Toxicity to Cells

Divide experiment into control group and test groups with different drug concentration. Digest cells, dilute to 200,000 cells/ml, transfer to culture plate, 100 μl per well for 96-well plate, incubate for 24 hours under 5% CO₂ at 37° C., ready for experiment when cells grown into monolayer. Dilute arginases with culture medium to 40 IU/ml, serial dilute to 20, 10, 5, 2.5 IU/ml and add into 96-well plates, 5 different concentrations altogether, 3 wells per concentration, change arginase solution every 4 days with the same original concentration. Observe cytopathological changes, 8 days or 4 days under microscope, totally destroyed is 4; 75% destroyed is 3; 50% destroyed is 2, 25% destroyed is 1; no changes is 0. Calculate TC50 and TC0 according to Reed-Muench Method:

$$TC 50 = Antilog ( B + \frac{50 - B}{A - B} \times C )$$

A=log>50% drug concentration; B=log<50% drug concentration; C=log times of dilution

### Example 3

### Test for Arginase Suppression of HBeAg and HBsAg

Experiment is designed to have HBsAg and HBeAg positive control group, negative control group, cell control group and test groups with different drug concentration. Grow 200000 cells/ml 2.2.15 cell on 24-well plate, 1 ml per well, incubate for 24 hours under 5% CO₂ at 37° C. Serial dilute TC0 drug solution into 5 dilutions for each drugs: 20, 10, 5, 2.5, 1.25 IU/ml for Arginase; 800, 400, 200, 100, 50 μg/ml for lamivudine. 4 wells per concentration, incubate under 5% CO₂ at 37° C., change drug solution every 4 days with the same concentration, retrieve culture medium at day 8, preserve at −20° C. Repeat experiment for 2 batches, test for HBsAg and HBeAg separately. Check cpm value for each wells using γ-counter.

Calculating drug effectiveness: calculate the mean and standard deviation of cpm from the cell control group and groups with different drug concentration, P/N value and percent suppression, IC50 and SI.
\textcircled{1}

$$percent antigen suppression ( \% ) = \frac{\begin{matrix}cell control cpm - \\ cpm of groups with drug\end{matrix}}{cell control cpm} \times 100$$

\textcircled{2} Calculate IC50 for drug suppression of antigen:

$$IC 50 = Antilog ( B + \frac{50 - B}{A - B} \times C )$$

A=log>50% drug concentration; B=log<50% drug concentration; C=log times of dilution
\textcircled{3} SI for Arginase in 2.2.15 cell culture towards HBsAg and HBeAg, calculated according to cellular pathological changes due to cytopathological toxicity.

$$SI = \frac{Cytopathological toxicity TC 50}{IC 50}$$

\textcircled{4} Calculate the cpm differences between HBsAg and HBeAg in different dilutions and control groups by t-test.

### Example 4

### Test for Arginase Suppression of 2.2.15 Cells DNA

Extraction of HBV-DNA from 2.2.15 cells supernatant: Grow 200000 cells/ml 2.2.15 cell on 24-well plate, 1 ml per well, add drugs after 24 hours incubation, change drug solution every 4 days with the same concentration, collect supernatant from cell culture after 8 days of incubation counted from the day drugs added into the culture, precipitate with polyethylene glycol, digest with proteinase K, extract with phenol:chloroform:isopentanol, nucleic acid precipitation by absolute ethanol and so on procedures, vacuum dry, re-dissolve in TE buffer as sample.

Dot blot: place dots: take 20 μl sample (contains 25 μg DNA), denature, neutralize, serial dilute 20×SSC buffer to 1:8 dilution on nitrocellulose membrane, oven dry, pre-hybridize, hybridize, wash membrane, radioactive self exposure and so on procedures. Develop X ray film with conventional method. Scan developed film with scanner, measure density with gel-pro software, calculate suppression rate and IC50.

$$HBV \text{-} DNA suppression in 2 \cdot 2 \cdot 15 cell culture medium = \frac{IOD - TIOD}{CIOD} \times 100 \%$$

Southern blot: Extraction of HBV-DNA from 2.2.15 cells: add drugs and incubate 2.2.15 cells for 8 days, remove medium and harvest cells, lyse cells with lysis solutions, extracts with equal volume phenol:chloroform:isopentanol twice, add absolute ethanol to precipitate nucleic acid, vacuum dry, re-dissolve in 20 μl TE buffer, add DNA sample buffer, put samples into agarose gel for electrophoresis. After electrophoresis, denature, neutralize and transfer to membrane. Oven dry, hybridize, expose with dot blotting the same time. Scan developed film with scanner, analyze relative density with gel-pro software, and calculate suppression rate and IC50.

### Results

Calculate TC50 and TC0 according to Reed-Muench Method. Calculate HBsAg and HBeAg suppression according to above mentioned formulas. Calculate suppression rate and IC50 by analyzing relative density of agarose gel electrophoresis of HBV-DNA.

1. Arginase Toxicity in 2.2.15 Cell Culture

To observe Arginase toxicity towards hepatitis B viral gene transfected human liver cancer 2.2.15 cells, add serial diluted drug solution into the cell culture after 24 hours of incubation. Starting from 40 IU/ml and subsequently 20, 10, 5, 2.5 IU/ml, change drug solution every 4 days until 8 days, observe cytopathological changes under microscope, and check for CPE with microscope. Results: Arginase toxicity in hepatitis B viral gene transfected human liver cancer cell 2.2.15 cells by CPE method (8 days drug administration): TC50 is 40 IU/ml and TC0 is 20±0 μg/ml in two batches experiments. Positive lamivudine control, TC50 is 1198.97±97.50 μg/ml, TC0 is 800±0 μg/ml (see Table. 1A).

2. Arginase Suppression of HBeAg and HBsAg

Add Arginase and lamivudine in TC0 concentration into 2.2.15 cells, check cpm value of HBsAg and HBeAg after 8 days, and calculate the effectiveness of drug suppression. See table 2 for experiment results.

2.1. Percent Arginase Suppression of HBeAg

Two batches Arginase experiments: Serial dilute TC0 20 IU/ml into 10, 5, 2.5 and 1.25 IU/ml, incubate 2.2.15 cells with each concentration for 8 days, average percent suppression of HBeAg in supernatant are: 20 IU/ml, 68.69±8.89% suppression; 10 IU/ml, 60.73±17.49% suppression; 5 IU/ml, 53.96±20.36% suppression; 2.5 IU/ml, 51.83±14.16% suppression; 1.25 IU/ml, 37.34% suppression. Average IC50 is 6.37±0.45 IU/ml, SI is 6.30±0.45.

2.2 Percent Arginase Suppression of HBsAg

First batch Arginase experiment: The suppression rate of HBsAg in cell culture supernatant of 2.2.15 cell culture after 8 days of incubation with the concentration of 20, 10, 5, 2.5, 1.25 IU/ml are 49.16%, 47.97%, 42.29% and 37.18% respectively. IC50 is 10.72 IU/ml, SI is 3.7.3. However, the percent suppression is low for the second batch. The suppression rate for HBsAg is below 50% with TC0 concentration equals to 20 IU/ml, IC50>20 IU/ml.

2.3. The Effect of Lamivudine on HBsAg and HBeAg

Serial dilute lamivudine from TC0 concentration 800 μg/ml to 400, 200, 100 and 50 μg/ml respectively and add into 2.2.15 cells, check HBsAg and HBeAg titer after 8 days of incubation, calculate the effect of suppression (See table 1B).

2.4. Percent Lamivudine Suppression of HBeAg

The average percent suppression of HBsAg in cell culture supernatant of 2.2.15 cell culture after 8 days of incubation with lamivudine in the concentration of 800, 400, 200, 100 and 50 μg/ml are: 8.23±3.02%, 12.99±0.46%, 17.83±2.09%, 15.84±2.33%, 14.10±1.27%. No significant suppression is shown.

2.5. Percent Lamivudine Suppression of HBsAg

The average percent suppression of HBeAg in cell culture supernatant of 2.2.15 cells after 8 days of incubation with lamivudine in the concentration of 800, 400, 200, 100 and 50 μg/ml are: 4.65±6.58%, 4.05±5.73%, 5.67±4.70%, 8.60±4.88%, 3.45±3.95%. No significant suppression is shown.

TABLE 1A
Arginase Suppression of HBsAg ad HBeAg in 2.2.15 cells (%)

| | | | | HBeAg (CPM) | | | | | |
|---|---|---|---|---|---|---|---|---|---|
| | | Day of | Drug | % | | | HBsAg (CPM) | | |
| | Experiment | drug | concen- | suppres- | IC50 | | % | IC50 | |
| Drug | batches | addition | tration | sion | IU/ml | SI | suppression | IU/ml | SI |
| Arginase | 1 | 8 | 20 | 74.9808 | 6.69 | 5.98 | 49.1558 | 10.72 | 3.73 |
| | | | 10 | 73.0985 | | | 47.9651 | | |
| | | | 5 | 68.3484 | | | 42.2871 | | |
| | | | 2.5 | 61.8387 | | | 37.1787 | | |
| | 2 | 8 | 20 | 62.4033 | 6.05 | 6.61 | 10.4731 | >20.00 | |
| | | | 10 | 48.3596 | | | 6.0761 | | |
| | | | 5 | 39.5618 | | | 1.4739 | | |
| | | | 2.5 | 41.8149 | | | 2.6073 | | |
| | | | 1.25 | 37.3426 | | | 4.463 | | |
| | Two batches | | 20 | 68.69 ± 8.89 | 6.37 ± 0.45 | 6.30 ± 0.45 | 29.81 ± 27.35 | 1^{st} batch | |
| | average | | 10 | 60.73 ± 17.49 | | | 27.02 ± 29.62 | 10.72 | |
| | | | 5 | 53.96 ± 20.36 | | | 21.88 ± 28.86 | 2^{nd} batch | |
| | | | 2.5 | 51.83 ± 14.16 | | | 19.92 ± 24.40 | >20 | |
| | | | 1.25 | 37.34 | | | 4.46 | Not even | |

TABLE 1B
Lamivudine Suppression of HBsAg ad HBeAg in 2.2.15 cells (%)

| | | | Drug | HBeAg (CPM) | | | HBsAg (CPM) | | |
|---|---|---|---|---|---|---|---|---|---|
| | | Day of | concen- | % | | | % | | |
| | Experiment | drug | tration | suppres- | IC50 | | suppres- | IC50 | |
| Drug | batches | addition | μg/ml | sion | μg/ml | SI | sion | μg/ml | SI |
| Lamivudine | 1 | 8 | 800 | 6.10 | >800 | | 0 | >800 | |
| | | | 400 | 12.67 | | | 8.10 | | |
| | | | 200 | 16.35 | | | 2.35 | | |
| | | | 100 | 17.48 | | | 12.05 | | |
| | | | 50 | 13.20 | | | 6.24 | | |
| | 2 | 8 | 800 | 10.37 | >800 | | 9.299 | >800 | |
| | | | 400 | 13.32 | | | 0 | | |
| | | | 200 | 19.31 | | | 8.99 | | |
| | | | 100 | 14.19 | | | 5.15 | | |
| | | | 50 | 14.99 | | | 0.65 | | |
| | Two batches | 8 | 800 | 8.23 ± 3.02 | >800 ± 0 TC0 800 | | 4.65 ± 6.58 | >800 ± 0 TC0 | |
| | average | | 400 | 12.99 ± 0.46 | μg/ml. | | 4.05 ± 5.73 | 800 μg/ml. | |
| | | | 200 | 17.83 ± 2.09 | No significant | | 5.67 ± 4.7 | No significant | |
| | | | 100 | 15.84 ± 2.33 | suppression | | 8.6 ± 4.88 | suppression | |
| | | | 50 | 14.10 ± 1.27 | shown. | | 3.45 ± 3.95 | shown. | |

3. Arginase and Lamivudine Suppression of HBV-DNA in Supernatant of 2.2.15 Cell Culture

3.1 Arginase Dot Blotting in HBV-DNA in Supernatant of 2.2.15 Cell Culture

The effect of arginase on HBV-DNA in supernatant of 2.2.15 cell culture, the IC50 of two batches of test solution against HBV-DNA after 8 days of incubation are 16.04, 10.31 IU/ml. average IC50 is 13.18±4.05 IU/ml, SI are 2.49, 3.88, average is 3.19±0.98. See Table 2 for result.

TABLE 2
Effect of Arginase on HBV-DNA in supernatant
of 2.2.15 cell culture

| | | | Dilution factor/percent suppression | | | |
|---|---|---|---|---|---|---|
| | | Drug | of HBV-DNA in cell culture supernatant | | | |
| | Day of | concen- | Original | % | | |
| | drug | tration | Solution | suppres- | IC50 | |
| Batch | addition | (μg/ml) | (IOD) | sion | (μg/ml) | SI |
| 1 | 8 | 20 | 1643.3 | 30.8113 | 16.04 | 2.49 |
| | | 10 | 1680.6 | 29.2409 | | |
| | | 5 | 2090.38 | 11.9877 | | |
| | | 2.5 | 1783.34 | 24.9152 | | |
| | | Control | 2375.1 | | | |
| 2 | 8 | 20 | 2430.14 | 47.7577 | 10.31 | 3.88 |
| | | 10 | 2881.48 | 38.0549 | | |
| | | 5 | 2613.11 | 43.8243 | | |
| | | 2.5 | 4118.31 | 11.466 | | |
| | | 1.25 | 3917.78 | 15.7769 | | |
| | | Control | 4651.67 | | | |

| | | |
|---|---|---|
| Two batches average | 13.18 ± 4.05 | 3.19 ± 0.98 |

3.2 Effect of Lamivudine to HBV-DNA in Supernatant of 2.2.15 Cell Culture

The effect of lamivudine to HBV-DNA in supernatant of 2.2.15 cell culture of first batch experiment: IC50 is 113.76 μg/ml, TC50 is 1198.97±97.50 μg/ml, SI is 10.54. See result in Table 3.

TABLE 3
Effect of Lamivudine on HBV-DNA in
supernatant of 2.2.15 cell culture

| | | | Dilution factor/percent suppression | | | |
|---|---|---|---|---|---|---|
| | | Drug | of HBV-DNA in cell culture supernatant | | | |
| | Day of | concen- | | % | | |
| | drug | tration | | suppres- | IC50 | |
| Drug | addition | (μg/ml) | CPM | sion | (μg/ml) | SI |
| Lamiv- | 8 | 800 | 663.013 | 82.5905 | 113.76 | 10.54 |
| udine | | 400 | 795.628 | 79.1083 | | |
| | | 200 | 1080.03 | 71.6404 | | |
| | | 100 | 1465.31 | 61.5237 | | |
| | | 50 | 2831.21 | 25.6576 | | |
| | | Control | 3808.34 | | | |

3.3. Arginase and Lamivudine Suppression of HBV-DNA Southern Blot in 2.2.15 Cells

3.3.1 The Suppression of HBV-DNA Southern Blot in 2.2.15 Cell by Arginase

Results show: The result of total HBV-DNA Southern Blot in 2.2.15 cells is totally suppressed after 8 days of incubation with Arginase added: two batches of experiment IC50 are 25.42, 14.17 IU/ml, average IC50 is 19.79±7.95, SI are 1.57 and 2.82 respectively, average is 2.19±0.88. The result of total HBV-DNA Southern Blot In Lane in 2.2.15 cells: two batches of experiment IC50 are 21.45, 18.67 IU/ml, average is 20.06±1.96 IU/ml, SI are 1.86, 2.14, average is 2.00±0.20. See results in Table 4.

TABLE 4
Arginase suppression of HBV-DNA Southern Blot in 2.2.15 cells

| | Day of | Drug | | | | | | | | |
|---|---|---|---|---|---|---|---|---|---|---|
| | drug | concen- | | % | | | In | % | | |
| | addic- | tration | Sum | Suppres- | IC50 | | Lane | Suppres- | IC50 | |
| Batch | tion | (μg/ml) | (IOD) | sion | μg/ml | SI | (IOD) | sion | IU/ml | SI |
| 1 | 8 | 20 | 25011 | 14.9893 | 25.42 | 1.57 | 31436 | 25.2982 | 21.45 | 1.86 |
| | | 10 | 24234 | 17.6303 | | | 32499 | 22.7722 | | |
| | | 5 | 20104 | 31.6679 | | | 28796 | 31.5717 | | |
| | | 2.5 | 21650 | 26.4131 | | | 28762 | 31.6525 | | |
| | | Control | 29421 | | | | 42082 | | | |
| 2 | 8 | 20 | 34433 | 47.9416 | 14.17 | 2.82 | 46760 | 38.6255 | 18.67 | 2.14 |
| | | 10 | 46884 | 29.1172 | | | 66241 | 13.0559 | | |
| | | 5 | 46283 | 30.0259 | | | 61655 | 19.0752 | | |
| | | 2.5 | 68619 | 0 | | | 88350 | 38.6255 | | |
| | | Control | 66143 | | | | 76188 | | | |

3.3.2 The Suppression of HBV-DNA Southern Blot in 2.2.15 Cells by Lamivudine

Results show: The suppression effect of total HBV-DNA Southern Blot in 2.2.15 cells with lamivudine: two batches of experiment IC50 are 84.27, 93.28 μg/ml, average is 88.78±6.37 μg/ml, TC50 is 1198.97 μg/ml, SI are 14.23 and 12.85 respectively, average is 13.54±0.97 (see Table 5).

TABLE 5
Lamivudine suppression of HBV-DNA
Southern Blot in 2.2.15 cells

| | | Drug | | | | |
|---|---|---|---|---|---|---|
| | Day of | concen- | | % | | |
| | drug | tration | Sum | Suppres- | IC50 | |
| Batch | addition | μg/ml | (IOD) | sion | μg/ml | SI |
| 1 | 8 | 800 μg/ml | 143.91 | 90.50 | 84.27 | 14.23 |
| | | 400 | 317.332 | 70.04 | | |
| | | 200 | 366.35 | 75.80 | | |
| | | 100 | 491.77 | 67.52 | | |
| | | 50 | 748.91 | 50.54 | | |
| | | Control | 1514.08 | | | |
| 2 | 8 | 800 μg/ml | 509.85 | 79.01 | 93.28 | 12.85 |
| | | 400 | 804.63 | 66.87 | | |
| | | 200 | 589.01 | 75.75 | | |
| | | 100 | 1002.21 | 58.74 | | |
| | | 50 | 710.239 | 70.76 | | |
| | | Control | 2428.92 | | | |

| | | |
|---|---|---|
| Two batches average | 88.78 ± | 13.54 ± |
| | 6.37 | 0.97 |

Discussion

The experiment observes the toxicity of Arginase and anti-hepatitis B virus positive control drug lamivudine on hepatitis B virus transfected human liver cancer cell 2215 cell line after 8 days of added drug incubation, the suppression of HBsAg and HBeAg secretion and in cell culture supernatant, and the suppression of HBV-DNA in cells. See Table 6 for summary.

TABLE 6
Summary of Effect of Arginase and Lamivudine on HBV-DNA in 2.2.15 cells

| | | | Cell | | |
|---|---|---|---|---|---|
| Cellular | | | Supernatant | | |
| toxicity | HBeAg | HBsAg | HBV-DNA | Cell HBV-DNA Southern Blot | |
| IU/ml | IC50 | IC50 | IC50 | IC50 | IC50 |

| Drugs | TC50 | TC0 | IU/ml | SI | IU/ml | SI | IU/ml | SI | μg/ml | SI | μg/ml | SI |
|---|---|---|---|---|---|---|---|---|---|---|---|---|
| Arginase | 40 | 20 ± 0 | 6.37 ± | 6.30 ± | \textcircled{1}10.72 | \textcircled{1}3.73 | 13.18 ± | 3.19 ± | 19.79 ± | 2.19 ± | 20.6 ± | 2.00 ± |
| | | | 0.45 | 0.45 | \textcircled{2}>20 | \textcircled{2}≦1 | 4.05 | 0.98 | 7.95 | 0.88 | 1.96 | 0.20 |
| Lamivudine | 1198.97 ± | 800 ± 0 | >800 | | | | >800 | | 113.76 | 10.54 | 88.78 ± | 13.54 ± |
| | 97.50 | μg/ml | μg/ml | | | | μg/ml | | | | 6.37 | 0.97 |

Annotation:
\textcircled{1}first batch,
\textcircled{2}second batch

1. Arginase Toxicity to 2.2.15 Cells

TC50 of Arginase is 40 IU/ml, TC0 is 20±0 IU/ml.

TC50 of positive control lamivudine is 1198.97±97.50 μg/ml; TC0 is 800±0 μg/ml.

2. Arginase and Lamivudine Suppression of the Secretion of HBsAg and HBeAg in 2.2.15 Cells

Serial dilute 4 concentrations of TC0 20 IU/ml Arginase and added into 2.2.15 cells to incubate for 8 days, the average suppression rate of two batches of experiments on HBeAg secretion is 68.69±8.89%, the IC50 to HBeAg is 6.37±0.45 IU/ml, SI is 6.30±0.45. The suppression rate of HBsAg is 29.81±27.35%, the IC50 to HBsAg are: first batch 10.72 IU/ml, SI is 3.73, second batch is 20 IU/ml.

Suppression rate is below 50%, IC50>20 IU/ml, SI:≦1. No average has been taken for the two batches of experiments.

No significant suppression action for HBeAg and HBsAg by adding TC0 800 μg/ml of lamivudine into 2.2.15 cell culture to incubate for 8 days. Half of the effective concentration and SI cannot be calculated.

3. Arginase and Lamivudine Suppression of HBV-DNA in 2.2.15 Cells

Results show: The IC50 of Arginase in HBV-DNA Dot Blot from supernatant of cell culture added with drug after 8 days of incubation is 13.18±4.05 IU/ml, SI is 3.19±0.98. The IC50 in HBV-DNA Southern Blot after 8 days is 19.79±7.95 IU/ml, SI is 2.19±0.88. The IC50 in HBV-DNA Dot Blot with added drug In Lane after 8 days is 20.06±1.96 μg/ml, SI is 2.00±0.20.

The IC50 of lamivudine in HBV-DNA Dot Blot is 113.76 μg/ml, SI is 10.54. In suppression of Southern blot, the IC50 of both batches of experiments are 84.27 and 93.28 μg/ml, average is 88.78±6.37 μg/ml, TC50 is 1198.97 μg/ml, SI are 14.23 and 12.85 respectively, average is 13.54±0.97

It must be noted that as used herein and in the appended claims, the singular forms “a” and “the” include plural references unless the context clearly dictates otherwise. Thus, for example, reference to “a pharmaceutical preparation” includes mixtures of different preparations and reference to “the method of treatment” includes reference to equivalent steps and methods known to those skilled in the art, and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference to describe and disclose specific information for which the reference was cited in connection with. The invention having been fully described, modifications within its scope will be apparent to those of ordinary skill in the art. All such modifications are within the scope of the invention.

Formulations of the pharmaceutical composition of the present invention can be used in the form of a solid, a solution, an emulsion, a dispersion, a micelle, a liposome, and the like, wherein the resulting formulation contains one or more of the modified human arginase in the practice of the present invention, as active ingredients, in a mixture with an organic or inorganic carrier or excipient suitable for enteral or parenteral applications. The active ingredients may be the arginase, for example, with the usual non-toxic, pharmaceutically acceptable carriers for tablets, pellets, capsules, suppositories, solutions, emulsions, suspensions, and any other form suitable for use in manufacturing preparations, in solid, semisolid, or liquid form. In addition auxiliary, stabilizing, thickening and coloring agents and perfumes may be used. The active ingredients of one or more arginase are included in the pharmaceutical formulation in an amount sufficient to produce the desired effect upon the target process, condition or disease.

Pharmaceutical formulations containing the active ingredients contemplated herein may be in a form suitable for oral use, for example, as tablets, troches, lozenges, aqueous or oily suspensions, dispersible powders or granules, emulsions, hard or soft capsules, or syrups or elixirs. Formulations intended for oral use may be prepared according to any method known in the art for the manufacture of pharmaceutical formulations. The tablets may be uncoated or they may be coated by known techniques to delay disintegration and absorption in the gastrointestinal tract, thereby providing sustained action over a longer period. They may also be coated to form osmotic therapeutic tablets for controlled release.

In some cases, formulations for oral use may be in the form of hard gelatin capsules wherein the active ingredients are mixed with an inert solid diluent, for example, calcium carbonate, calcium phosphate, kaolin, or the like. They may also be in the form of soft gelatin capsules wherein the active ingredients are mixed with water or an oil medium, for example, peanut oil, liquid paraffin, or olive oil.

The pharmaceutical formulations may also be in the form of a sterile injectable solution or suspension. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,4-butanediol. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, or synthetic fatty vehicles, like ethyl oleate, or the like. Buffers, dextrose solutions preservatives, antioxidants, and the like, can be incorporated or used as solute to dissolve the soluble enzyme as required.

The pharmaceutical formulations may also be an adjunct treatment together with other chemotherapeutic agents.

What is claimed is:

1. The use of an arginine degrading enzyme in the manufacture of a medicament for the treatment of hepatitis.

2. The use according to claim 1, wherein said enzyme is an isolated and substantially purified recombinant arginase.

3. The use according to claim 1, wherein the purity of said recombinant arginase is 80-100%.

4. The use according to claim 3, wherein said recombinant arginase is human arginase I.

5. The use according to claim 3, wherein said recombinant arginase is arginine deiminase.

6. The use according to claim 4, wherein said enzyme comprising substantially the same nucleic acid sequence as set forth in SEQ ID NO: 1 or SEQ ID NO: 2, wherein said nucleic acid sequence comprising substantially the same amino acid sequence as set forth in SEQ ID NO: 3.

7. The use according to claim 4, wherein said enzyme having a specific activity of 250 I.U./mg.

8. The use according to claim 4, wherein said enzyme comprising a modification that results in having sufficient stability and an in vitro plasma half-life of at least approximately 3 days.

9. The use according to claim 8, wherein said enzyme is pegylated.

10. The use according to claim 9, wherein said pegylation results from covalently attaching at least one polyethylene glycol (PEG) moiety to said arginase using a coupling agent.

11. The use according to claim 10, wherein said coupling agent is 2,4,6-trichloro-s-triazine (cyanuric chloride, CC) or succinimide propionic acid (SPA).

12. The use according to claim 4, wherein said human arginase I comprising six histidines attached to the amino terminal end thereof.

13. The use according to claim 1, wherein said hepatitis is hepatitis B.

14. A pharmaceutical composition comprising arginine degrading enzyme.

15. The pharmaceutical composition of claim 14, wherein said enzyme is an isolated and substantially purified recombinant arginase.

16. The pharmaceutical composition of claim 15, wherein the purity of said recombinant arginase is 80-100%.

17. The pharmaceutical composition of claim 15, wherein said recombinant arginase is human arginase I.

18. The pharmaceutical composition of claim 15, wherein said recombinant arginase is arginine deiminase.

19. The pharmaceutical composition of claim 17, wherein said enzyme comprising substantially the same nucleic acid sequence as set forth in SEQ ID NO: 1 or SEQ ID NO: 2, wherein said nucleic acid sequence comprising substantially the same amino acid sequence as set forth in SEQ ID NO: 3.

20. The pharmaceutical composition of claim 17, wherein said enzyme having a specific activity of 250 I.U./mg.

21. The pharmaceutical composition of claim 17, wherein said enzyme having a half-life of at least 3 days in patient plasma.

22. The pharmaceutical composition of claim 17, wherein said enzyme having a half-life of at least 1 days in patient plasma.

23. The pharmaceutical composition of claim 17 wherein said enzyme is modified by pegylation.

24. The pharmaceutical composition of claim 17, wherein said human arginase I comprising six histidines attached to the amino terminal end thereof.

25. The pharmaceutical composition of claim 14, wherein said enzyme reduces the physiological arginine level in patients.

26. The pharmaceutical composition of claim 14, wherein said enzyme modulates hepatitis.

27. The pharmaceutical composition of claim 26, wherein said hepatitis is hepatitis B.

28. The pharmaceutical composition of claim 14, wherein said composition can be further manufactured in the form of a solid, a solution, an emulsion, a dispersion, a micelle, or a liposome.

29. The pharmaceutical composition of claim 14, wherein said composition is suitable for oral use or injection.


SEQUENCE LISTING (9 sequences)

SEQ ID NO: 1 - DNA, 2002 bp, Homo sapiens
gaattgtacgtcaaagagatgaagcagaaaaacgtcgtcgagaagaagctgaacgacaaaaagtgaaatgcgagggaagtccaagaaatggtgattatgagggtgtctatttcaccaaaaacggagaatatttattggaattaagagtctctgggactgctcttgtaaatgctccttgtaatttaaaggatattgacataacgaaatggttgtgtaaaacagggagattatatcttgataaggttaagaaatttgaaatagttactattctttcccatgacgtagaaaatcaaaagattataacagaatgggagtcactccccagagaggctttacccgaacaatttgattcataagaactaattagtagcgctttccaatggaggcgcttttttatttgggtagttgcataccactaaagatgttcaggtgcacatgagcattggaggaaaggaacgctttagggggaagggaaacctttaaacagtcttaatcccccttgattttatgttctctgtaaactgcgtccggtaaatctcaggatagacaatcggcggttaacggcttgagtgcgggggcagtttagaaagaatatgattggagggattcatagatgcatcaccatcaccatcatatgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcacaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgtgtccatgtcattcaaaaaatgtgattctaga

SEQ ID NO: 2 - DNA, 990 bp, Homo sapiens
  CDS (1)..(990)
atgcatcaccatcaccatcatatgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcacaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaa

SEQ ID NO: 3 - protein, 329 aa, Homo sapiens
MHHHHHHMSAKSRTIGIIGAPFSKGQPRGGVEEGPTVLRKAGLLEKLKEQECDVKDYGDLPFADIPNDSPFQIVKNPRSVGKASEQLAGKVAQVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 4 - protein, 7 aa, Artificial
  His Tag
MHHHHHH

SEQ ID NO: 5 - DNA, 33 bp, Artificial
  primer
ccaaaccatatgagcgccaagtccagaaccata

SEQ ID NO: 6 - DNA, 39 bp, Artificial
  primer
ccaaactctagaatcacattttttgaatgacatggacac

SEQ ID NO: 7 - DNA, 24 bp, Artificial
  primer
ctctggccatgccagggtccaccc

SEQ ID NO: 8 - DNA, 969 bp, Homo sapiens
  CDS (1)..(969)
atgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcacaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaa

SEQ ID NO: 9 - protein, 322 aa, Homo sapiens [sequence omitted]